(19) United States
(12) Patent Application Publication
(10) Pub. No.: US 2023/0181894 A1
(43) Pub. Date: Jun. 15, 2023

(54) CONTROLLER FOR ARTIFICIAL HEART AND METHOD

(71) Applicant: Scandinavian Real Heart AB, Västerås (SE)

(72) Inventors: Azad NAJAR, Västerås (SE); Nils BRYNEDAL IGNELL, Västerås (SE)

(21) Appl. No.: 17/912,317

(22) PCT Filed: Mar. 18, 2021
(86) PCT No.: PCT/EP2021/057036

§ 371 Date: Sep. 16, 2022

(30) Foreign Application Priority Data
Mar. 18, 2020 (SE) 2050298-5

(51) Int. Cl. A61M 60/148; A61M 60/531

(52) U.S. Cl. CPC A61M 60/148; A61M 60/531; A61M 2205/3331

(57) ABSTRACT

The invention relates to a controller unit (100) and method for controlling a cardiac prosthesis (200). The prosthesis comprising: at least one pump portion (202, 203, 602, 702); an inlet (210, 610, 710) connected to said at least one pump portion; an outlet (213, 613, 713) connected to said at least one pump portion; a pressure sensor (231; 232) configured to measure pressure of a fluid flowing from the inlet to the outlet; a pump actuator (221, 222) configured to induce the flow of the fluid flow. The controller unit further comprises a memory and a processing unit, wherein the controller unit is configured to: obtain a pressure value from the pressure sensor, obtain a desired value for the pressure of the fluid flowing into the pump, calculate an error signal equal to the difference of desired value for the pressure and the measured pressure, and control the output of the pump such that the measured pressure is near or equal to the desired pressure, by controlling a pump stroke rate and/or a pump stroke volume.

### TECHNICAL FIELD

Generally, the present invention relates to control methods and devices for artificial hearts and particularly to a response of an artificial heart system to varying physiological demand and includes mechanisms accommodating the actual flow imbalance between pulmonary and systemic circulations.

### BACKGROUND

Despite steady progress in developing a permanent artificial heart for long-term implantation in a patient as a substitute for a failed natural heart, a number of issues must still be resolved. Among the issues that need to be addressed in an untethered artificial heart system are control strategies that respond to varying physiological demand, and mechanisms for accommodating the flow imbalance between the pulmonary and systemic circulations.

Left-right cardiac output differences have been well documented. Physiologically, the blood flow pumped by the left side of the heart is higher than that pumped by the right side of the heart. This difference is largely attributable to a circulatory pathway known as the bronchial shunt. This flow originates in the left arterial system, passes through the bronchial tissue, and then returns directly to the left atrium. This difference typically appears to be up to about ten percent of cardiac output with the left side flow always greater than the right-side flow. Artificial heart systems must account for this inherent physiological circulatory imbalance. In addition, sources of flow imbalance can be fabricated. For example, differences in regurgitation through artificial valves provided on the left and right sides can introduce a flow imbalance. Artificial heart systems must account for these types of circulating imbalances as well.

A blood-pumping device is disclosed in WO 2016/020219, by same applicant, comprising at least a first pump and a second pump and a left and right pump actuating means for inducing a blood flow in a body's circulatory system is disclosed. Each pump comprises one upper chamber having an inlet channel and one lower chamber having an outlet channel. The upper and lower chambers are separated by a movable valve plane provided with a valve. The pump actuating means are configured to apply a movement to said valve plane in an upward and downward direction between said upper and lower chambers in response to control signals from a control unit, such that when said valve plane moves in an upward direction the valve provided in the valve plane is in an open position allowing a flow of blood from the upper chamber to the lower chamber, and when the valve plane moves in a downward direction the valve is in the closed position and blood is ejected from the lower chamber through the outlet channel. The bottom part of the lower chamber is provided with a bag-like portion.

FIG. 1 discloses schematics of the blood pumping device 1 of WO 2016/020219, in a cross-sectional view, having four chambers. The four- chambered blood-pumping device 1 comprises two pumps, a first pump 2 and a second pump 3, and a first and second pump actuator for inducing a blood flow in a body's circulatory system. The first and the second pumps 2, 3 are identical in their construction. Each pump comprises an upper chamber 9 and a lower chamber 12. The upper chamber 9 has an inlet channel (not shown), which allows blood to enter the upper chamber 9. The upper chamber corresponds to an atrium of the natural heart. The lower chamber 12 is provided with an outlet channel (not shown), which allows blood to exit the lower chamber 12. The lower chamber 12 corresponds to a ventricle of the natural heart. Using a movable valve plane 7, the upper and lower chambers 9 and 12 are separated. The valve plane corresponds to the atrioventricular (AV) plane (i.e., the plane of fibrous tissue) between the atria and ventricles of a natural heart. A valve 14 is arranged in the valve plane 7, which corresponds to the tricuspid valve or the mitral valve depending on whether it is located in a pump, which acts in the pulmonary circuit or in the aortic circuit. The bottom part of the lower chamber 12 is advantageously designed to have a shape that is similar to the anatomic shape of the ventricles in the natural heart. In the four- chambered blood-pumping device 1, the bottom part of the lower chamber 12 has a bag-like shape designed to mimic the internal shape of the ventricle in a natural heart. The blood flow (arrow) which passes from the upper chamber 9 through the valve 14 and into the lower chamber 12 hits a stopping surface at the bottom part of the bag-like shape and comes to a sudden stop, at which the flow abruptly changes direction and continues along the outlet channel (not shown). The turn at the inside of the bag-like portion at the bottom of the lower chamber 12 forms a bend of approximately 90-340°, more preferably between 100-300°, more preferably between 105-200°, and most preferably a bend between 110-150°, which is similar to the bend inside the ventricle of a natural heart. Thereafter the blood continues into the outlet channel. The cross section of the bag-like portion at the bottom part of the lower chamber 12 advantageously has a triangular shape to enable an optimal flow of blood from the lower chamber 12 into the outlet channel. As in a natural heart, the triangular shaped cross section facilitates the formation of flow channels inside the cavity of the lower chamber 12, such that blood will arrive from different angles at the stopping surface of the bag-like portion, stop, change direction, enter the outlet channel and subsequently leave the blood-pumping device through an outlet valve. Alternatively, the cross section of the inside construction of the lower chamber 12 may have an oval or circular cross section. The inner walls at the bottom of the lower chamber 12, as well as the outlet channels are advantageously provided with a rough surface to simulate the trabeculae carinae i.e., the muscular ridges that crisscross and project from the inner walls of the ventricles of a natural heart. Said rough surface is lined by ridges and protrusions which protrude approximately 0.01-3 mm, preferably at least 0.5-2 mm from the lower chamber 12 surface. The outlet channel and bottom of the lower chamber 12 may also have smooth surfaces. The outlet channel from the lower chamber 12 may also have a diameter, which decreases continuously similarly to the design of the outlet of a ventricle in a natural heart.

WO 2017/137486, by same applicant, relates to a blood pump housing device designed to enclose and protect a total artificial heart when implanted in a subject. The blood-pump housing device comprises a first and second artificial heart pump receiving part configured to receive and partly enclose a first and a second artificial heart pump of a Total Artificial Heart (TAH), and a first and second pump actuation enclosing part configured to partly house a first and second pump actuation means. The artificial heart pump receiving parts and pump actuation means enclosing parts are arranged to connect to each other in a leak-free manner.

### SUMMARY

There is a need for a control device and method that automatically controls the heart rate and stroke volume of at least each blood pump device in general, and the previously mentioned pump devices in particular.

The present invention also provides for a response of an artificial heart system to varying physiological demand and includes mechanisms accommodating the actual flow imbalance between pulmonary and systemic circulations.

For these reasons, a method for controlling a cardiac prosthesis is provided. The cardiac prosthesis comprises: at least one pump portion; an inlet connected to the at least one pump portion; an outlet connected to the at least one pump portion; a pressure sensor configured to measure pressure of a fluid flowing from the inlet to the outlet; a pump actuator configured to induce the flow of the fluid flow, and a controller unit. The method comprises the steps of: obtaining a pressure value from the pressure sensor, obtaining a desired value for the pressure of the fluid flowing into the pump, calculating an error signal equal to the difference of desired value for the pressure and the measured pressure, and controlling the output of the pump such that the measured pressure is near or equal to the desired pressure, by controlling the pump actuator to control a pump stroke rate and/ or a pump stroke volume. In one embodiment, the fluid is blood. The output may be a cardiac output. According to one embodiment, the pump comprises a chamber corresponding to one of a right or left atrium.

In one embodiment, the cardiac prosthesis may comprise two similar pumps. The two pumps are connected to a systemic and pulmonary circulations, respectively, and both controlled individually, by setting a limit to the cardiac output of the pump connected to the pulmonary circulation. Thus, the method comprises further steps of: obtaining the cardiac output of the pump connected to the systemic circulation, given the cardiac output of the pump connected to the systemic circulation, setting a limit to the cardiac output to which the pump connected to the pulmonary circulation, wherein the limit is updated as the cardiac output of the pump connected to the systemic circulation changes; and providing a control signal to said pump actuator.

In one embodiment, the two pumps are connected to the systemic and pulmonary circulations, respectively, having a desired cardiac output, attaining stroke volumes and stroke rate of the two pumps, the method comprising the steps of: attaining desired cardiac output for either pump, using the desired cardiac output of the two pumps, finding a stroke rate, given the attained stroke rate, attain a stroke volume for either pump, such that the product of the stroke rate and stroke volume equals each desired cardiac output; and providing a control signal to said pump actuator.

In one embodiment, the pressure sensor is arranged in communication with said chamber. In alternative embodiment, the pressure is measured in a position between in opening of the inlet and an end of the chamber forming an atrium. One embodiment comprises measuring pressure inside the thorax cavity as reference pressure.

In one embodiment, the controller unit is configured to detect if the atrial pressure becomes low or a thoracic pressure increases, i.e., a suction event, and act to prevent low atrial pressure or thoracic pressure increase. Thus, the control unit compares the atrial pressure received from the pressure sensor, averaged throughout a course of one stroke, to a desired atrial pressure and if the average of the atrial pressure falls too far below the desired pressure a suction event is detected. In a further step, the atrial pressure is averaged throughout the course of a period in order to reduce noise and prevent false detection of a suction even and once a suction event is detected by the control unit, arranging an inactive period of time during which suction event are not detected. The method further comprises, once a suction event is detected, a stepwise increase in the desired atrial pressure is executed in order to prevent further suction events and generate an alert.

The invention also relates to a controller unit for controlling a cardiac prosthesis. The prosthesis comprises: at least one pump portion; an inlet connected to said at least one pump portion; an outlet connected to said at least one pump portion; a pressure sensor configured to measure pressure of a fluid flowing from the inlet to the outlet; a pump actuator configured to induce the flow of the fluid flow, a memory and a processing unit. The controller unit is configured to: obtain a pressure value from the pressure sensor, obtain a desired value for the pressure of the fluid flowing into the pump, calculate an error signal equal to the difference of desired value for the pressure and the measured pressure, and control the output of the pump such that the measured pressure is near or equal to the desired pressure, by controlling a pump stroke rate and/or a pump stroke volume. In one alternative embodiment the controller unit comprises functional blocks of: flow control, configured to determine a correct flow limit and a desired atrium pressures, as input to keep a cardiac output within a range; cardiac output determination, determining the cardiac output of either pump to control atrium pressures, determining a heart rate: wherein the heart rate and the cardiac output of either pump provides the stroke volume for either pump.

In one embodiment, the cardiac prosthesis comprises a first and a second pump, and the controller unit is configured to keep the flow of the first pump low enough, so that the second pump does not reaches its maximum flow. In an alternative embodiment, the cardiac prosthesis comprises two similar pumps and the two pumps are connected to a systemic and pulmonary circulations, respectively, and both controlled individually, by setting a limit to the cardiac output of the pump connected to the pulmonary circulation, controller unit being further configured to: obtain the cardiac output of the pump connected to the systemic circulation, given the cardiac output of the pump connected to the systemic circulation, set a limit to the cardiac output to which the pump connected to the pulmonary circulation, wherein the limit is updated as the cardiac output of the pump connected to the systemic circulation changes; and provide a control signal to the pump actuator.

In one embodiment, the cardiac prosthesis comprises two similar pumps and the two pumps are connected to a systemic and pulmonary circulations, respectively, having a desired cardiac output, attaining stroke volumes and stroke rate of the two pumps, the controller unit being further configured to: attain a desired cardiac output for either pump, use the desired cardiac output of the two pumps, to calculate a stroke rate, given the attained stroke rate, attain a stroke volume for either pump, such that the product of the stroke rate and stroke volume equals each desired cardiac output; and provide a control signal to said pump actuator.

According to one embodiment, the controller unit comprises a signal receiver to receive signals and detect if the atrial pressure becomes low or a thoracic pressure increases, i.e., a suction event, and act to prevent low atrial pressure or thoracic pressure increase. In one embodiment, the processing unit of the controller unit is configured to compare the atrial pressure received from the pressure sensor, averaged throughout a course of one stroke, to a desired atrial pressure and if the average of the atrial pressure falls too far below the desired pressure a suction event is detected. In another embodiment, the processing unit of the controller unit is configured to average the atrial pressure throughout the course of a period in order to reduce noise and prevent false detection of a suction even and once a suction event is detected by the control unit, arranging an inactive period of time during which suction event are not detected. In one embodiment, once a suction event is detected, the controller unit is configured to a stepwise increase in the desired atrial pressure in order to prevent further suction events and generate an alert.

The invention also relates to a cardiac prosthesis comprising a controller unit as described above.

The invention further relates to a pressure sensor for use in a cardiac prosthesis. The prosthesis comprising: a housing having an upper portion and a body, an inlet, an outlet, a chamber between the inlet and the outlet. The pressure sensor comprises: a flexible membrane covering an open portion in said upper portion or body, a pressure transferring medium, a pipe comprising said pressure transferring medium, and a pressure sensitive sensor.

The invention also relates to a cardiac prosthesis comprising such a pressure sensor.

### BRIEF DESCRIPTION OF THE DRAWINGS

Reference is made to the attached drawings, wherein elements having the same reference number designation may represent like elements throughout.

FIG. 1 is a view of a four-chambered blood-pumping device according to prior art;

FIG. 2 is a schematic view of a control device according to the present invention connected to a blood-pumping device;

FIG. 3 is a functional block scheme of a control unit in accordance with the invention;

FIGS. 4a and 4b are schematic views of a PID-controller in accordance with one aspect of the invention;

FIG. 5 is a graph of exemplary ratios between heart rate and blood flow;

FIG. 6 is a perspective view of an artificial heart according to one embodiment;

FIG. 7a is a cross-sectional view of a pump of the artificial heart according to one embodiment of the present invention and FIG. 7b is enlarged view of encircled portion in FIG. 7a;

FIG. 8 is a schematic view of a control unit according to the present invention;

FIG. 9 illustrates, in a perspective view, an embodiment of one pump constating the part of the heart prothesis; and 10 illustrates a cross-sectional view of the pump of FIG. 9; and

FIG. 11 is a cross-sectional and very schematic view of thorax and thorax cavity.

### DETAILED DESCRIPTION

The following detailed description refers to the accompanying drawings. The same reference numbers in different drawings may identify the same or similar elements.

The term artificial heart as used herein relates to a pumping device connectable to a subject.

The term “stroke volume” as used herein, relates to a specific amount of fluid displaced under a specific time interval and the term “heart rate” as used herein relates to a rate a pump displaces an amount of fluid.

The terms “right” or “left atrium” as used herein, relate to a compartment inside the pump housing into which a fluid is provide and discharged from.

In the following, a control device and method that sets a heart rate and stroke volume of either half of an artificial heart, in particular such as the ones described in WO 2016/020219 or WO 2017/137486 is described. However, it should be clear that the device and the method according to the present invention may be applied to any pumping device having a chamber through which a fluid can pass. The controllers and methods described herein may also be used in heart assistant pumps with necessary modifications.

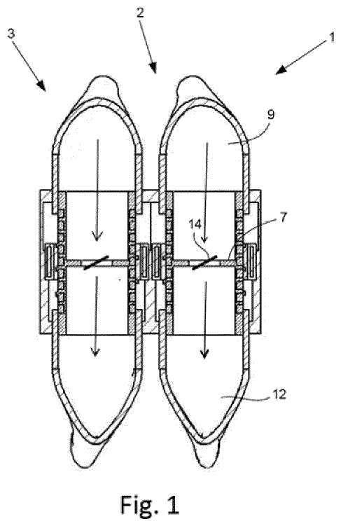

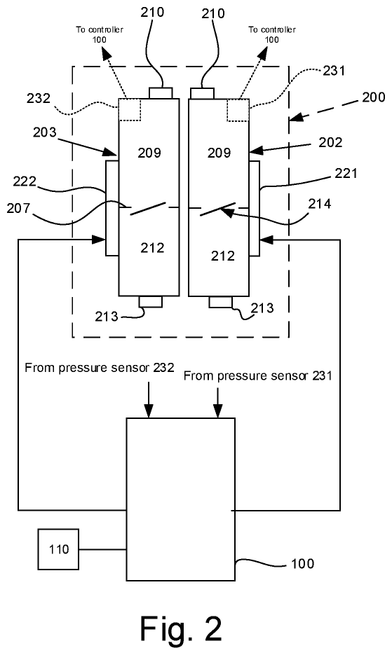

FIG. 2 illustrates a very schematic control unit 100 and a blood-pump device 200 in accordance with one exemplary embodiment of the present invention. The blood-pump 200 comprises a first pump 202 and a second pump 203, and a first and a second pump actuating means 221, 222 for inducing a blood flow. Each pump comprises one upper chamber 209 having an inlet channel 210 and one lower chamber 212 having an outlet channel 213. The upper and lower chambers are separated by a movable valve plane 207 provided with a valve 214. Each pump-actuating means 221, 222 is configured to apply a movement to the valve plane 207 in an upward and downward direction between the upper and lower chambers in response to control signals from the control unit 100, such that when the valve plane 207 moves in an upward direction, the valve 214 is in an open position allowing a flow of blood from the upper chamber 209 entering from the inlet 210 to the lower chamber 212, and when the valve plane moves in a downward direction the valve is in the closed position and blood is ejected from the lower chamber 212 through the outlet channel 213. Preferably, a bottom part of the lower chamber 212 is provided with a bag-like portion (not shown but illustrated schematically in FIG. 1).

The actuating means 221 and 222 may comprise magnetic or electromagnetic motor or any other suitable driving system, which can receive a signal and drive or displace pumping portion directly or in cooperation with gearbox (not shown).

The blood-pump system comprises pressure sensors 231 and 232. The sensor and its function are described in more detail below. The pressure sensors are configured to measure the average of the atrial pressures throughout each heartbeat. This averaging may help to reduce any noise in the measured signals.

The pressure inside the thorax cavity continuously changes during the respiration. As the pressure inside the thorax cavity affects the pressure inside the atrium (209), it is desirable to measure the pressure inside the thorax cavity (reference pressure) as well. This way, it is possible to compensate for the altering of the pressure inside the thorax cavity. The pressure inside the thorax cavity can be measured by:
1. Using a pressure sensor assembled inside the pump but designed to measure the pressure inside the thorax cavity,
2. Using a separate pressure sensor placed inside the thorax cavity and connected to the pump by a wire, or
3. Arranging a pressure sensor outside the thorax cavity but inside the chest-wall and exactly behind the pleura layer (the most inner layer in the chest wall).

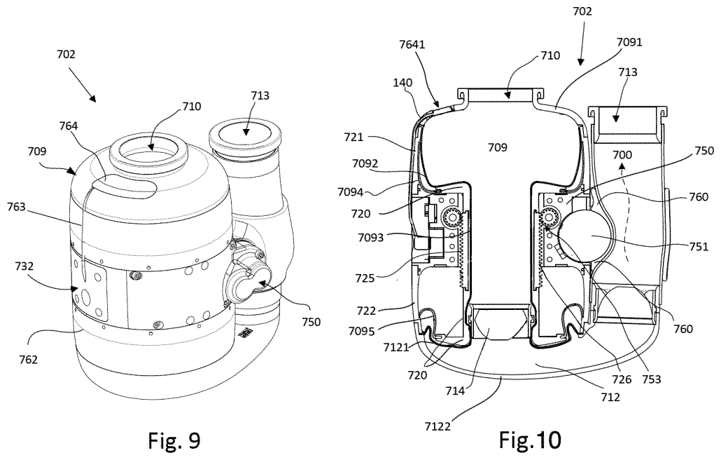

Thus, the pressure sensor can be connected to the pleura (pleural) membrane, which will function as a natural flexible membrane between the thorax cavity and the pressure sensor. It is enough to use one pressure sensor for measuring the pressure inside the thorax cavity for both the right and left pump to remove any effect of breathing and the measurement is done outside of the heart itself. This is illustrated in FIG. 11.

FIG. 11 is a cross-sectional and very schematic view of a human thorax and thorax cavity.

Reference sign 501 designates a rib, 502 skin, 503 superficial fascia, 504 intercostal muscles, 505 parietal pleura, 506 pleural cavity, 507 visceral pleura and 508 the lung.

In this application, the pressure sensors 510 are arranged between ribs 501 and in contact with parietal pleura 507 to measure pressure in pleural cavity 506. The electrical connections, which may be extended out through the tissues are not illustrated. The pressure sensor may also be arranged in vicinity of costal cartilage. The pressure sensors 510 function in similar way as described pressure sensor but the electronics, sensor element, flexible membrane and pressure transferring medium are arranged in one housing.

The control unit 100 has input signals from the pressure sensors 231 and 232 and output signal to pump-actuating means 221, 222. The signals may be received and provided directly or via interfaces, wirelessly or wired. A power source 110 may be connected to or integrated in the control unit 100.

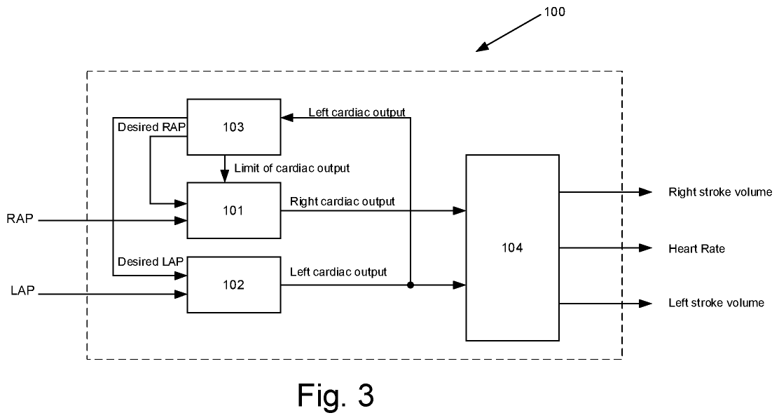

FIG. 3 illustrates the schematics and functional blocks of an exemplary control unit 100.

The control unit 100, according to this embodiment, may comprise functional blocks:
a cardiac output control for right atrium 101,
a cardiac output control for left atrium 102,
a flow control block 103, and
a processing block 104.

The main function of the control unit 100 is to set a heart rate and stroke volumes for either half (i.e., left and right pumps) of the blood-pump 200 to values that are appropriate to the current physiological state of the subject.

The control problem may be solved by dividing it into sub-problems, comprising:
Flow control: This determines correct flow limit and desired atrium pressures, which will be inputs to keep the cardiac output within appropriate ranges. Desired atrial pressures can be overridden, by allowing the operator of the prothesis to set desired atrium pressures manually.
Determining cardiac output of either pump to control atrium pressures: This is done individually with no inter-dependency between the pumps.
Determining a heart rate: The heart rate and the cardiac output of either pump (calculated in the previous step) will in turn provide the stroke volumes for either pump.

The inputs to the functional block include:
Right Atrium Pressure (RAP), measured by the pressure sensor 231 in the right atrium to (right) cardiac output control 101; and
Left Atrium Pressure (LAP), measured by the pressure sensor 232 in the left atrium to (left) cardiac output control 102.

Determining a cardiac output of either pump may be carried out with only the atrium pressures as input.

The stroke volumes, as mentioned, may be calculated as stroke length multiplied with a constant, and may hence not agree with the actual pumped stroke volumes. This implies that the flows reported also are only approximations.

The functional block flow control 103 determines the flow limit and the desired atrium pressures. The flow limit is a boundary for the cardiac output of the right pump.

The objective of the flow control is to keep the flow of the right-side pump 202 low enough, so that the left pump 203 never reaches its maximum flow. If this happens, i.e., a maximum flow is reached, the left pump cannot keep LAP bounded, which would pose a risk of pulmonary edema. Limiting the right pump's capacity may cause RAP to rise; this is however can be seen as acceptable. Higher RAP will also increase Central Venous Pressure (CVP), which will help reducing venous return. The flow control block 103 receives output from left cardiac output control 102 and sets desired RAP, desired LAP, and limit of right-side cardiac output.

The actual flow of either half of the pump, i.e., 202 and 203, may be dependent on various factors such as outflow and inflow pressures, even given the same heart rate and stroke length. Because of this, the flow limit of the right pump (202) cannot be set constant but has to be changed dynamically.

The following parameters may be considered:

Flow state: The flow state is a variable determined by the flow of the left pump 203. Table 1 shows some exemplary values for different flow states and left pump flow in liter/minute:

TABLE 1

| | Left pump flow |
|---|---|
| Flow state | (l/min) |
| Extreme | >6.3 |
| High | 6-6.3 |
| Normal | 5-6 |
| Low | <4 |

Flow limit: The flow limit is the maximum allowed cardiac output of the right pump. The following rules may apply:
When transitioning from a Low or Normal flow state to a High or Extreme state (see Table 1), the flow limit may be set equal to the current right cardiac output. This is the only state transition that directly results in changing the flow limit.
When in a High flow state, the flow limit is constant.
When in an Extreme flow state, the flow limit is reduced at a constant rate, though it is not set lower than half of the maximum cardiac output.
When in a Low or Normal flow state: If, and only if, the right pump's output is limited by the flow limit, the flow limit is increased at a constant rate.
The flow limit may be reduced at a faster rate than it is increased.
The limit should never be set higher than the maximum cardiac output.
The limit should never be set lower than half of the maximum cardiac output.

Desired Atrium pressure: At low flow states, the desired atrium pressures may be reduced in order to help improve venous return.

During a low flow state, the desired atrium pressures may be determined by the left flow and may be set according to Table 2, which shows exemplary values of desired left and right atrium pressures (in mmHg) with respect to the left flow (liter/minute):

TABLE 2

| | Desired LAP | Desired RAP |
|---|---|---|
| Left flow (l/min) | (mmHg) | (mmHg) |
| >5 | 9 | 7 |
| 4-5 | 7 | 5 |
| <4 | 5 | 3 |

If Low flow or Extreme flow states are detected an alert may be generated.

Consequently, the flow control block 103 receives output form left cardiac output control 102 and sets desired RAP, desired LAP and limit of right-side cardiac output.

The cardiac output control for the right-side pump 101 receives RAP, desired RAP as well as the limit to right-side cardiac output and outputs the right-side pump's cardiac output. The cardiac output control for the left-side pump 102 receives LAP as well as desired LAP and outputs the left-side pump's cardiac output. This is further described below.

The processing block 104 uses right and left cardiac output levels to generate correct stroke volume, heart rate and left stroke volume, which are provided by the controller to the pump actuating means.

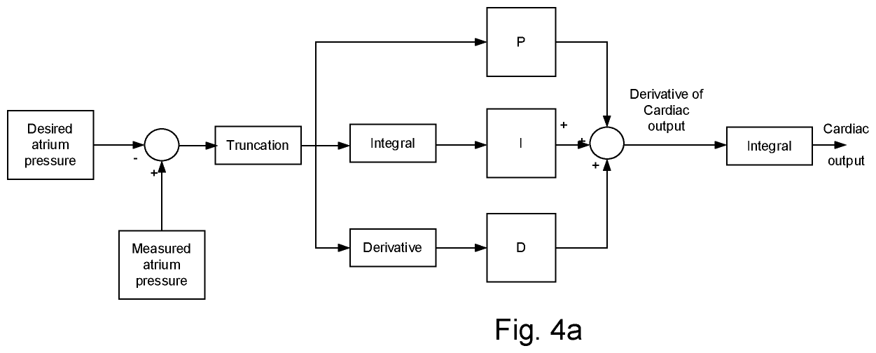

In one embodiment, the cardiac output is controlled by a proportional—integral—derivative (PID) controller that controls the derivative of the cardiac output in order to keep the actual atrium pressure close to (preferably equal to) the desired atrium pressure. This is schematically illustrated in FIG. 4a.

FIG. 4b is a simplified PID-controller diagram in which it is assumed that derivatives and integrals cancel out. This removes the need for a numeric derivation, which could introduce noise. The PID controller continuously calculates an error value e(t) as the difference between a desired set point (SP) and a measured process variable (PV) and applies a correction based on proportional (P), integral (I), and derivative (D) terms. Consequently, the SP is the desired atrium pressure and PV is the cardiac output. The measured atrium pressure is subtracted from the desired atrium pressure to create the error value e(t). The calculated error signal e(t) is truncated before being fed to the PID-controller. This is done so that the controller will not “over-react” if large differences between desired and actual atrial pressures are present, which may occur transiently. The error is simply multiplied by P, I and D actions. Then the resulting “error control actions” are added together, integrated and output as cardiac output (left or right).

In one embodiment, it is possible to connect the two pumps 202 and 203 to systemic and pulmonary circulations, respectively. Both can individually be controlled, by setting a limit to the cardiac output of the pump connected to the pulmonary circulation. Consequently, the cardiac output of the pump connected to the systemic circulation is obtained and given the cardiac output of the pump connected to the systemic circulation, an appropriate limit to the cardiac output, the pump connected to the pulmonary circulation is obtained, and the limit is updated as the cardiac output of the pump connected to the systemic circulation changes.

In another embodiment, two pumps may be connected to the systemic and pulmonary circulations, respectively, each having a desired cardiac output. Appropriate stroke volumes and stroke rate of the two pumps are attained; desired cardiac output for either pump is obtained; using the desired cardiac output of the two pumps, an appropriate stroke rate is found, and given the attained stroke rate, attain appropriate stroke volumes for either pump, such that the product of the stroke rate and either stroke volume equals either desired cardiac output.

The heart rate portion integrated in functional block 104 takes two inputs, the left and right cardiac outputs, Q_{left} and Q_{right}, and outputs the heart rate (HR) as well as the left and right stroke volumes, SV_{left} and SV_{right}. A solution to the following system of equations is needed:

Q_{left}=HR*SV_{left}

Q_{right}=HR*SV_{right}

The heart rate procedure is limited by a few factors:
Max heart rate,
Min heart rate,
Max stroke volume,
Min stroke volume,

This means that there is a maximum and minimum cardiac output.

When the flows (Q_{left} and Q_{right}) are small, the stroke volumes shall be small as well. Only when the flows are large, the stroke volume shall be set closer to the maximum.

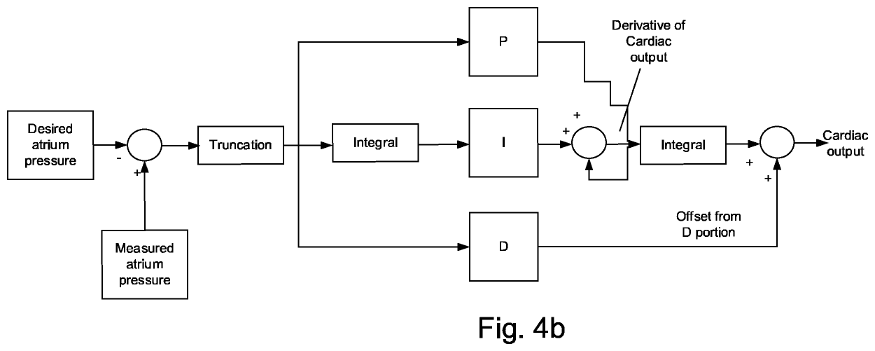

FIG. 5 shows a graph of heart rate (beats/min)and flow (liters/min), which allows to interpret the problem as finding two points on the same height (same heart rate) and at flows equal to Q_{left} and Q_{right}.

To simplify terminology, Q_{big} is the maximum of Q_{left} and Q_{right} and Qₛₘₐₗₗ is the minimum.

A function f may be used, as shown in FIG. 5, to get the heart rate to a reasonable heart rate for Qₛₘₐₗₗ. The function f is only given as an example and other functions may be utilized. The curve f(Q) in FIG. 5 may stay high even relatively far to the left. This means that during times when the flows are low (which may happen if the patient has a small blood volume due to, for example, dehydration or bleeding) the heart rate is kept relatively high and the stroke volumes are kept low, this constitutes a form of tachycardia.

The heart rate HR will be:

HR=f(Qₛₘₐₗₗ)

giving:

SVₛₘₐₗₗ=Qₛₘₐₗₗ/HR

SV_{big}=Q_{big}/HR

unless this requires SV_{big}>SVₘₐₓ in which case, heart rate is set according to the biggest flow:

HR=Q_{big}/SVₘₐₓ

The above values, heart rate and stroke volumes, are truncated to stay within their bounds.

The controller unit 100 converts HR and SV values to control signals and directly or indirectly provides them to the actuating means 221 and 222.

The controller unit 100 may be an implanted or integrated microcomputer or electronic chips. The microcomputer can provide the control signals to the pump actuating means to change its pumping activity. If for some reason the microcomputer is not receiving any input information, the pump actuating means may continue at a constant level of activity.

In some circumstances, the atrial pressure may become too low, or the thoracic pressure may increase (e.g., because of assisted ventilation or other reasons). In presence of such situations, the atrium and/or connected veins might collapse. This may, fully or partially, occlude the inflow of blood into the pump (this may happen to either pump, individually). This is known as a suction event.

The controller unit of the present invention can detect these events and act to prevent them.

A suction event is detected by the control unit, by comparing the atrial pressure received from the pressure sensor, averaged throughout the course of one stroke, to the desired atrial pressure. If the average of the atrial pressure falls too far below the desired pressure (by a certain margin, which may be in the order of tens of mmHg), a suction event is detected.

The atrial pressure is averaged throughout the course of a period (e.g., in the order of half a second to a second) in order to reduce noise and prevent false positives (detecting a suction even when there was none). Once a suction event is detected by the control unit, there will be an inactive period of time (in the order of seconds) during which suction events will not be detected. This is to avoid the same suction event from being detected several times.

The exact values for the detection margin for atrial pressure and the length of the inactive period can be set to different values. The detection margin would be in the order of tens of mmHg and the inactive period would be in the order of a few seconds.

Once a suction event is detected, a stepwise increase in the desired atrial pressure (in the order of one or a few mmHg) may be executed in order to prevent further suction events. It is possible to appropriately select the increase in desired atrial pressure.

Upon detection of suction events, they may be reported to a user interface of the device.

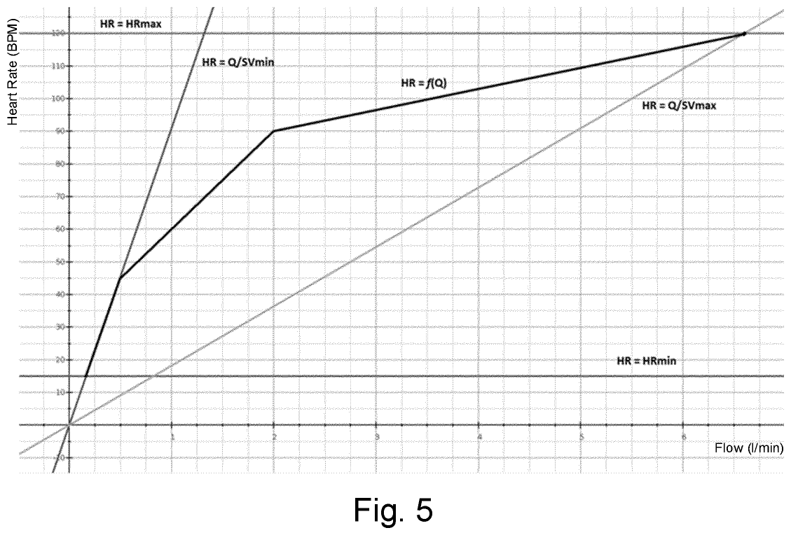

FIG. 6 illustrates one of the pumps 602 of an artificial heart (prothesis), according to one embodiment of the invention, comprises a cylindrical housing 660, an upper chamber 609 inside the housing, an inlet channel 610, an outlet channel 613, a pump actuator housing 661, a pressure sensor housing 662, a pressure transferring medium pipe 663, and a sensor covers 664.

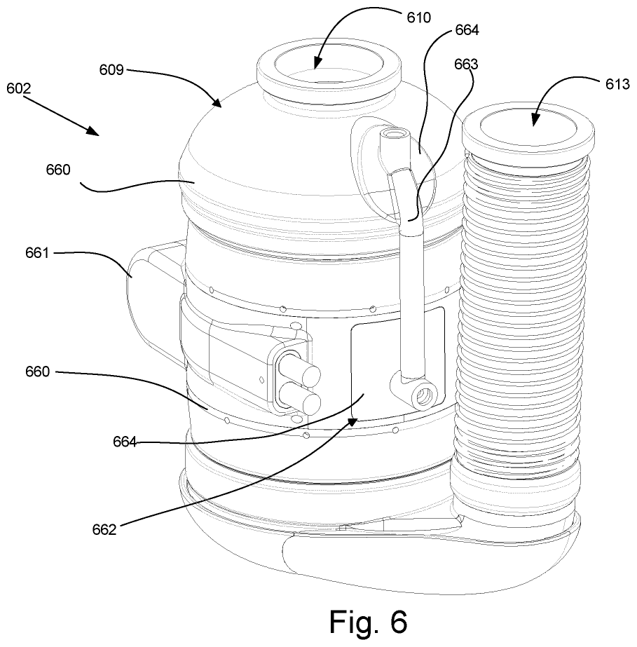

FIG. 7a is a cross sectional schematic view of an alternative pump 702. FIG. 7b is an enlarged view of the encircled section comprising the sensor 732.

The sensor construction 732 comprises an opening, i.e., a reservoir 738 in the upper chamber's 709 wall, a flexible thin membrane 733, a pipe 763, an electrical sensor 734, a circuit board 735, pressure transferring medium 736, and attachment arrangement 737.

The sensor 734 for sensing pressure may comprise a MEMS (small-scale microelectromechanical system) sensor arranged on the electrical board 735 (PCB) together with corresponding electronics. The pipe 763 may comprise a metallic cylinder or similar and is attached to the electronic circuit from one side in such a way that the lumen 7631 of the pipe is directly connected and is in communication with the MEMS sensor 734. The pressure sensor pressure receiving portion is arranged in the left atrium 709 and right pump atria (or upper half of each pump) as described earlier. The lumen 7631 of the pipe 763 is in connection with the flexible membrane 733 of the atrial wall 709 of each respective pump directly or through another cylindrical structure (assembling tube) in order to prolong the length of the lumen to make the assembling of the pump together with the pressure sensor possible and satisfactory. The flexible membrane 733 is arranged as a portion of the inner wall of the housing and portion of a reservoir 738 containing the pressure transferring medium 736. The wall of the assembling tube may be made of a hard plastic or metallic material. The connection of the pipe lumen or the lumen of the assembling tube 763 to the membrane 733 of the atrial wall is sealed in a leakage-free way, e.g., using an adhesive agent or a sealing washer. The pipe 763 and the reservoir 738 may be filled with a biocompatible, implantable oil, such as medical graded silicone oil, as the pressure transferring medium 763. The reservoir 738 may be part of the pipe 763 having same dimensions or slightly different. There may be means assembled to the metallic pipe lumen or the lumen of the assembling pipe to facilitate the filling of the lumen with oil and there may be other means connected to the metallic cylinder lumen or the lumen of the assembling tube to facilitate the discharge of the air during the filling of the lumen with the pressure transferring medium.

In operation, when the membrane 733 of the atrial wall 760 is affected by the pressure of the blood inside the atrium 709 of each pump, the flexible membrane 733 will either bulge when the pressure inside the atrium increases or it will bow inwards to atrial cavity when the pressure decreases. In this way, the pressure transfer medium inside the reservoir and lumen of the pipe 763 compresses or will expand due to the influence of the pressure inside the atrium 709. The pressure is transferred inside the pipe 763 from the membrane to the sensor 734 surface. The sensitive surface of the MEMS sensor 734 is thus affected by the pressure changing in the pressure transfer medium and the MEMS sensor translates the pressure to a digital value and an electrical signal representing the pressure is generated and provided to the controller unit 100.

The construction of sensor assembly, according to this exemplary embodiment, is to gain space and use the available space in the pump housing. Thus, the above sensor structure is one example of a sensor and other sensors may also be used in the pump, e.g., the entire sensor can be arranged in the atrium wall. The presser measurement can also be carried out between the inlet opening and end of the chamber forming the atrium.

Many of previously exemplified embodiments relate to a pulsating pump or a displacement pump; however, the method and control unit of the invention are likewise applicable in systems having different types of pump(s), e.g., a centrifugal pump. The centrifugal pump may include one or several of canned, radial, side channel, regenerative turbine, axial, diagonal pump type. A displacement pump may include one or several of dosing, progressing cavity, gear, multi screw, piston diaphragm, plunger and piston, rotary lobe, vacuum and hose pump type.

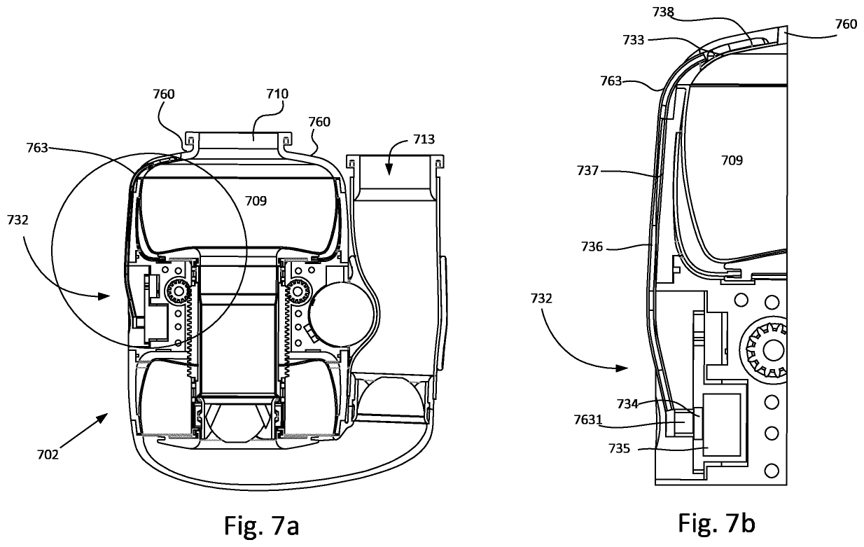

FIG. 8 is a diagram of an exemplary controller unit 100 in which methods described herein may be implemented. The controller unit 100 may include a bus 110, a processor 120, a memory 130, a read only memory (ROM) 140, a storage device 150, an input device 160, an output device 170, and a communication interface 180. Bus 110 permits communication among the components of the controller unit 100. The controller unit 100 may also include one or more power supplies (not shown). One skilled in the art would recognize that the controller unit 100 may be configured in a number of other ways and may include other or different elements.

The processor 120 may include any type of processor or microprocessor that interprets and executes instructions. Processor 120 may also include logic that is able to decode media files and generate output to, for example, to a speaker, a display, etc. Memory 130 may include a random-access memory (RAM) or another dynamic storage device that stores information and instructions for execution by processor 120. Memory 130 may also be used to store temporary variables or other intermediate information during execution of instructions by processor 120.

ROM 140 may include a conventional ROM device and/or another static storage device that stores static information and instructions for processor 120. Storage device 150 may include a magnetic disk, solid state drive or optical disk and its corresponding drive and/or some other type of recording medium and its corresponding drive for storing information and instructions. Storage device 150 may also include a flash memory (e.g., an electrically erasable programmable read only memory (EEPROM)) device for storing information and instructions.

Input device 160 may include one or more conventional mechanisms that permit a user to input information to the controller unit 100, such as a keyboard, a keypad, a directional pad, a mouse, a pen, voice recognition, a touchscreen and/or biometric mechanisms, etc. Output device 170 may include one or more conventional mechanisms that output information to the user, including a display, a printer, one or more speakers, etc. Communication interface 180 may include any transceiver-like mechanism that enables controller unit 100 to communicate with other devices and/or systems. For example, communication interface 180 may include a modem or an Ethernet interface to a LAN. Alternatively, or additionally, communication interface 180 may include other mechanisms for communicating via a network, such as a wireless network. For example, communication interface may include a radio frequency (RF) transmitter and receiver and one or more antennas for transmitting and receiving RF data.

The controller unit 100, consistent with the invention, provides a platform through which a as described earlier. According to an exemplary implementation, the controller unit 100 may perform various processes in response to processor 120 executing sequences of instructions contained in memory 130. Such instructions may be read into memory 130 from another computer-readable medium, such as storage device 150, or from a separate device via communication interface 180. It should be understood that a computer-readable medium may include one or more memory devices or carrier waves. Execution of the sequences of instructions contained in memory 130 causes processor 120 to perform the acts that have been described earlier. In alternative embodiments, hard-wired circuitry may be used in place of or in combination with software instructions to implement aspects consistent with the invention. Thus, the invention is not limited to any specific combination of hardware circuitry and software.

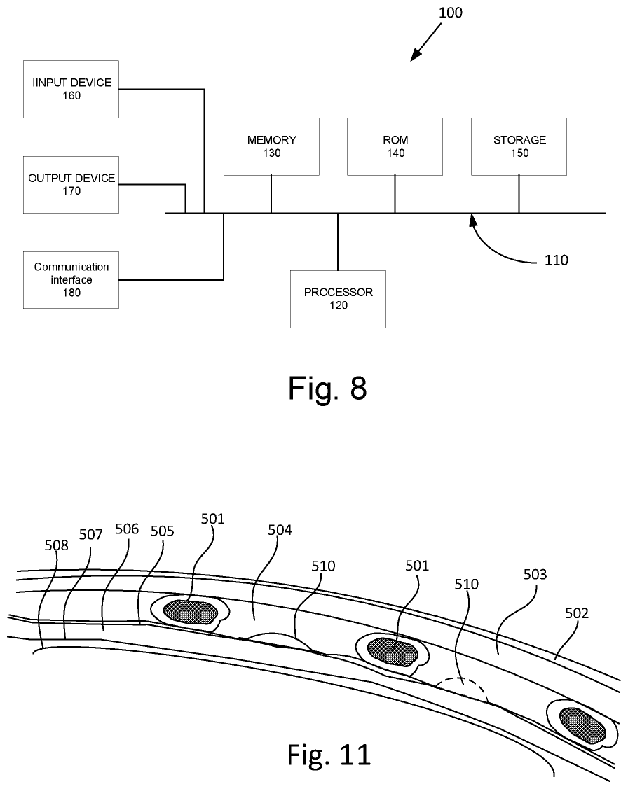

FIGS. 9 and 10 illustrate, in perspective, the pump of FIG. 7a which is a further enhancement of the prior art pump by the same applicant and thus a part of an enhanced artificial heart. FIG. 10 is a cross-sectional view of FIG. 9. This embodiment comprises a first blood receiving part 702 of the artificial heart and the drive/actuator system 750 to drive/actuate the artificial heart to create the pumping mechanism. The artificial heart is mainly consisting of two pumps 702 the left pump and the right pump (not shown). Each pump comprises one blood receiving part and one driver/actuator system. The blood receiving part is assembled to the drive system in an easy way by screws, glue or in combination. The blood receiving part comprises an artificial atrium 709, and an artificial ventricle 712. Between the artificial atrium 709 and artificial ventricle 712 there is a connecting cylinder 720 with it is one-way valve corresponding to the mitral valve in left side of the natural heart and to the tricuspid valve in the right side of the natural heart. The connecting cylinder can be made of flexible blood compatible material such as polyurethane, silicone or any other blood compatible material.

Each atrium 709 consists mainly of an atrial covering wall, which mostly consists of two layers: a stiff outer layer and a flexible inner layer. The stiff outer layer 7091, is advantageously made of hard polyurethane, hard silicone, biocompatible metal such as titanium or stainless steel or any other biocompatible stiff material. The inner layer is a flexible blood compatible membrane 7092 made of polyurethane, silicone or any other blood compatible material. The inner flexible membrane is an extension of a flexible atrial membrane, which is an extension of the flexible membrane 7093 lined the inner surface of the connecting AV-cylinder 720, which is an extension of a flexible ventricular membrane 7121. Further there is an atrial protective flexible membrane 7094 which protects the atrial flexible membrane 7092. Further, there is an atrial assembling ring 721, which is a stiff and made of stiff polyurethane, stiff silicone, biocompatible metal such as titanium or stainless steel or any other biocompatible firm material. The atrial assembling ring assembled to the upper edge of the drive/actuating system by screws, glue or in combination. The atrium has an inlet opening 710 to let the blood flowing inside the atrium. There is a pressure window 7641 with a diameter of, e.g., at least 5-30 mm with a circular, oval, or any other shape. The wall of the pressure window consists only of the flexible membrane without the stiff wall layer. This pressure window is a part of the pressure sensor construction, as described above.

Each ventricle 712 consists mainly of a ventricular covering wall, which mostly consists of two layers an outer layer, which is stiff and made of stiff polyurethane, stiff silicone, biocompatible metal such as titanium or stainless steel or any other biocompatible stiff material. Further, there is an inner flexible blood compatible membrane 7121 made of polyurethane, silicone or any other blood compatible material. The inner flexible membrane is an extension of a flexible ventricular membrane, which is an extension of the flexible membrane 7093 lined the inner surface of the connecting AV-cylinder 720. Further there is a ventricular protective flexible membrane 7095 which protects the atrial flexible membrane 7121. Further, there is a ventricular assembling ring 722, which is a stiff and made of stiff polyurethane, stiff silicone, biocompatible metal such as titanium or stainless steel or any other biocompatible stiff material. The ventricular assembling ring assembled to the lower edge of the drive/actuating system by screws, glue or in combination.

The driver/actuator system 750 consists of, in the housing 762, a gearbox 753 and an electrical motor 751. The housing is made of a stiff plastic biocompatible material such as PEEK and any other biocompatible plastic material or biocompatible metal. The housing encloses the gearbox, and the electrical motor. The gearbox consists of multiple cogwheels and metal axels. The electrical motor is of type of brushless motor or any other type of electrical motor with or without encoder. The motor 751 is arranged inside the pump housing between the outlet 713 and the pump. A spacer 760 may be arranged between the outlet 713 housing and the motor 751. The spacer 760 is made of a material with good thermal transfer capacity, such that when blood (or other liquid) flows 700 through the outlet pipe, the heat from the driving mechanism is transferred by means of the spacer 760 and the wall of the outlet 713 to the blood, which transfers and reduces the heat from the driving mechanism.

The center of the drive/actuation system has cylindrical shape and enclosing AV-cylinder 720 which consists of a stiff plastic biocompatible material such as stiff polyurethane, stiff silicone, or a biocompatible metal such as titanium, stainless steel or any other biocompatible stiff material. The AV-cylinder is lined by a flexible membrane 7093, which is an extension of the flexible atrial membrane 7092 and flexible ventricular membrane 7021. The AV-cylinder encloses valve 714. There are two racks, 725 and 726, one on each side of the AV-cylinder 720. Each rack is articulated with the cogwheel of the gearbox 753 to be actuated in upward and downward direction.

There may be a wire (not shown), which is connected to the drive/actuation system to supply the electrical motor with electrical power and control signal. Further there may be a covering membrane (not shown), which is a layer of biocompatible plastic material such as polyurethane or silicone enclose the whole drive/actuation system allowing the wire to the electrical motor and the pipe of the pressure senor to penetrate through this covering membrane.

It should be noted that the word “comprising” does not exclude the presence of other elements or steps than those listed and the words “a” or “an” preceding an element do not exclude the presence of a plurality of such elements. It should further be noted that any reference signs do not limit the scope of the claims, that the invention may be implemented at least in part by means of both hardware and software, and that several “means”, “units” or “devices” may be represented by the same item of hardware.

The above mentioned and described embodiments are only given as examples and should not be limiting to the present invention. Other solutions, uses, objectives, and functions within the scope of the invention as claimed in the below described patent claims should be apparent for the person skilled in the art.

The various embodiments of the present invention described herein is described in the general context of method steps or processes, which may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), Solid State Drive, etc. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Software and web implementations of various embodiments of the present invention can be accomplished with standard programming techniques with rule-based logic and other logic to accomplish various database searching steps or processes, correlation steps or processes, comparison steps or processes and decision steps or processes. It should be noted that the words “component” and “module,” if used herein and in the following claims, is intended to encompass implementations using one or more lines of software code, and/or hardware implementations, and/or equipment for receiving manual inputs.

The foregoing description of embodiments of the present invention, have been presented for purposes of illustration and description. The foregoing description is not intended to be exhaustive or to limit embodiments of the present invention to the precise form disclosed, and modifications and variations are possible in light of the above teachings or may be acquired from practice of various embodiments of the present invention. The embodiments discussed herein were chosen and described in order to explain the principles and the nature of various embodiments of the present invention and its practical application to enable one skilled in the art to utilize the present invention in various embodiments and with various modifications as are suited to the particular use contemplated. The features of the embodiments described herein may be combined in all possible combinations of methods, apparatus, modules, systems, and computer program products.

1. A method for controlling a cardiac prosthesis, the cardiac prosthesis having:
at least one pump portion;
an inlet connected to the at least one pump portion;
an outlet connected to the at least one pump portion;
a pressure sensor configured to measure pressure of a fluid flowing from the inlet to the outlet;
a pump actuator configured to induce the flow of the fluid flow, and
a controller unit;
the method comprising the following steps performed by the controller:
obtaining a pressure value from the pressure sensor,
obtaining a desired value for the pressure of the fluid flowing into the pump,
calculating an error signal equal to the difference of desired value for the pressure and the measured pressure, and
controlling the output of the pump such that the measured pressure is near or equal to the desired pressure, by controlling the pump actuator to control at least one of a pump stroke rate or a pump stroke volume.

2. The method of claim 1, wherein the fluid is blood.

3. The method of claim 1, wherein the output is cardiac output.

4. The method of claim 1, wherein the pump comprises a chamber corresponding to one of a right or left atrium.

5. The method of claim 1, wherein the cardiac prosthesis comprises two similar pumps.

6. The method of claim 5, wherein each of said two pumps are connected to a systemic and pulmonary circulations, respectively, and both controlled individually, by setting a limit to the cardiac output of the pump connected to the pulmonary circulation, the method comprising further steps of:
obtaining the cardiac output of the pump connected to the systemic circulation,
given the cardiac output of the pump connected to the systemic circulation, setting a limit to the cardiac output to which the pump connected to the pulmonary circulation, wherein the limit is updated as the cardiac output of the pump connected to the systemic circulation changes; and
providing a control signal to said pump actuator.

7. The method of claim 5, wherein said two pumps are connected to the systemic and pulmonary circulations, respectively, having a desired cardiac output, attaining stroke volumes and stroke rate of the two pumps, the method comprising the steps of:
attaining desired cardiac output for either pump,
using the desired cardiac output of the two pumps, finding a stroke rate,
given the attained stroke rate, attain a stroke volume for either pump, such that the product of the stroke rate and stroke volume equals each desired cardiac output; and
providing a control signal to said pump actuator.

8. The method of claim 4, wherein the pressure sensor is arranged in communication with said chamber.

9. The method according to claim 1, wherein the pressure is measured in a position between in opening of the inlet and an end of the chamber forming an atrium.

10. The method according to claim 1, further comprising measuring pressure inside the thorax cavity as reference pressure.

11. The method according to claim 1, the controller unit is configured to detect if the atrial pressure becomes low or a thoracic pressure increases, and act to prevent low atrial pressure or thoracic pressure increase.

12. The method of claim 11, wherein the control unit compares the atrial pressure received from the pressure sensor, averaged throughout a course of one stroke, to a desired atrial pressure and if the average of the atrial pressure falls too far below the desired pressure a suction event is detected.

13. The method of claim 12, wherein the atrial pressure is averaged throughout the course of a period to reduce noise and prevent false detection of a suction event and once a suction event is detected by the control unit, arranging an inactive period of time during which suction event are not detected.

14. The method of claim 12, wherein once a suction event is detected, a stepwise increase in the desired atrial pressure is executed to prevent further suction events and generate an alert.

15. A controller unit for controlling a cardiac prosthesis, the prosthesis comprising:
at least one pump portion connected to an inlet and an outlet; and
a pump actuator configured to induce a flow of a fluid,
the controller unit comprising:
a memory, and
a processing unit, configured to:
receive a pressure value from the pressure sensor, the pressure sensor configured to measure pressure of the fluid flowing from the inlet to the outlet,
receive a desired value for the pressure of the fluid flowing into the pump,
calculate an error signal equal to the difference of desired value for the pressure and the measured pressure, and
control the output of the pump such that the measured pressure is near or equal to the desired pressure, by controlling at least one of a pump stroke rate or a pump stroke volume, wherein the pump stroke rate and a cardiac output of the at least one pump portion provides the pump stroke volume for the at least one pump portion.

16. The controller unit of claim 15, comprising functional blocks of:
flow control, configured to determine a correct flow limit and a desired atrium pressures, as input to keep a cardiac output within a range;
cardiac output determination, configured to determine the cardiac output of the at least one portion to control atrium pressures,
a pump stroke rate determination.

17. The controller unit of claim 15, wherein the cardiac prosthesis comprises a first and a second pump, and the controller unit is configured to keep the flow of the first pump low enough, so that the second pump does not reaches its maximum flow.

18. The controller unit according to claim 15, wherein the cardiac prosthesis comprises two similar pumps, each of which is connected to a systemic and pulmonary circulations, respectively, and both pumps are controlled individually, by setting a limit to the cardiac output of the pump connected to the pulmonary circulation, controller unit being further configured to:
obtain the cardiac output of the pump connected to the systemic circulation,
given the cardiac output of the pump connected to the systemic circulation, set a limit to the cardiac output to which the pump connected to the pulmonary circulation, wherein the limit is updated as the cardiac output of the pump connected to the systemic circulation changes; and
provide a control signal to the pump actuator.

19. The controller unit according to claim 15, wherein the cardiac prosthesis comprises two similar pumps and the two pumps are connected to a systemic and pulmonary circulations, respectively, having a desired cardiac output, attaining stroke volumes and stroke rate of the two pumps, the controller unit being further configured to:
attain a desired cardiac output for either pump,
use the desired cardiac output of the two pumps, to calculate a stroke rate,
given the attained stroke rate, attain a stroke volume for either pump, such that the product of the stroke rate and stroke volume equals each desired cardiac output; and
provide a control signal to said pump actuator.

20. The controller unit according to claim 15, comprising a signal receiver to receive signals and detect if the atrial pressure becomes low or a thoracic pressure increases, and act to prevent low atrial pressure or thoracic pressure increase.

21. The controller unit of claim 20, wherein the processing unit of the controller unit is configured to compare the atrial pressure received from the pressure sensor, averaged throughout a course of one stroke, to a desired atrial pressure and if the average of the atrial pressure falls too far below the desired pressure a suction event is detected.

22. The controller unit of claim 21, wherein the processing unit of the controller unit is configured to average the atrial pressure throughout the course of a period in order to reduce noise and prevent false detection of a suction event and once a suction event is detected by the control unit, arranging an inactive period of time during which suction event are not detected.

23. The controller unit of claim 22, wherein, once a suction event is detected, the controller unit is configured to a stepwise increase in the desired atrial pressure to prevent further suction events and generate an alert.

24-26. (canceled)

27. The controller unit according to claim 15, wherein the fluid is blood.

28. The controller unit according to claim 15, wherein the pump comprises a chamber corresponding to one of a right or a left atrium.

29. The controller unit according to claim 28, wherein the pressure sensor is arranged in communication with said chamber.

30. The controller unit according to claim 15, wherein the pressure sensor is configured to measure pressure in a position between opening of the inlet and an end of the chamber forming an atrium.

31. The controller unit according to claim 15, wherein the sensor is configured to measure pressure inside a cavity of pump representing a thorax cavity as reference pressure.

32. A cardiac prosthesis comprising a controller unit for controlling the cardiac prosthesis, the prosthesis comprising:
at least one pump portion connected to an inlet and an outlet; and
a pump actuator configured to induce the flow of the fluid,
a memory, and
a processing unit, configured to:
receive a pressure value from a pressure sensor configured to measure pressure of the fluid flowing from the inlet to the outlet;
receive a desired value for the pressure of the fluid flowing into the pump;
calculate an error signal equal to the difference of desired value for the pressure and the measured pressure; and
control an output of the pump such that the measured pressure is near or equal to the desired pressure, by controlling a pump stroke rate and a pump stroke volume, wherein the pump stroke rate and a cardiac output of the at least one pump portion provides the pump stroke volume for the at least one pump portion.

33. A pressure sensor configured to be connected to a controller unit configured to control a pump stroke rate and a pump stroke volume of a cardiac prosthesis, the pressure sensor comprising:
a flexible membrane covering an open portion of a pump of the cardiac prosthesis,
a pressure transferring medium,
a pipe containing the pressure transferring medium, and
a pressure sensitive sensor configured to communicate with said controller unit.

34. A cardiac prosthesis comprising a pressure sensor configured to be connected to a controller unit configured to control a pump stroke rate and a pump stroke volume of the cardiac prosthesis, the pressure sensor comprising:
a flexible membrane covering an open portion of a pump of the cardiac prosthesis,
a pressure transferring medium,
a pipe containing the pressure transferring medium, and
a pressure sensitive sensor configured to communicate with said controller unit.

35. A controller unit for controlling a cardiac prosthesis, the prosthesis comprising:
two pump portions connected to an inlet and an outlet; and
pump actuators configured to induce a flow of a fluid,
the controller unit comprising:
a memory, and
a processing unit, configured to:
receive a pressure value from a pressure sensor configured to measure pressure of the fluid flowing from the inlet to the outlet;
receive a desired value for the pressure of the fluid flowing into the pump,
calculate an error signal equal to the difference of desired value for the pressure and the measured pressure,
control an output of the pump such that the measured pressure is near or equal to the desired pressure, by controlling a pump stroke rate and a pump stroke volume, wherein the pump stroke rate and a cardiac output of the at least one pump portion provides the pump stroke volume for the at least one pump portion; a flow control, configured to determine a correct flow limit and a desired atrium pressure, as input to keep a cardiac output within a range;
a cardiac output determination, configured to determine the cardiac output of the at least one pump portion to control atrium pressures, and
a pump stroke rate determination.
